(19) United States
(12) Patent Application Publication
(10) Pub. No.: US 2023/0035112 A1
(43) Pub. Date: Feb. 2, 2023

(54) APPLIANCE FOR MONITORING THE COMPRESSION THERAPY PROVIDED BY A COMPRESSION MEANS

(71) Applicant: KOB GMBH, Wolfstein (DE)

(72) Inventor: Ferdinand TAMOUÉ, Ludwigshafen am Rhein (DE)

(21) Appl. No.: 17/784,177

(22) PCT Filed: Nov. 16, 2020
(86) PCT No.: PCT/EP2020/082177

§ 371 Date: Jun. 10, 2022

(30) Foreign Application Priority Data
Dec. 16, 2019 (DE) 10 2019 134 538.3

(51) Int. Cl. A61B 5/00; A61B 5/053

(52) U.S. Cl. CPC A61B 5/4848; A61B 5/6802; A61B 5/053; A61B 2562/0209

(57) ABSTRACT

An appliance (10) for monitoring the compression therapy provided by a compression means comprises a measuring device made of a textile material for placing on a wearer and having two end portions (20, 22) which enclose between them a central portion (28), wherein two electrodes (12-18) are arranged in each end portion (20, 22) and each form an electrode pair with an electrode (12-18) of the other end portion (20, 22), wherein one electrode pair determines a flow of electric current and the other electrode pair determines an electrical voltage between the electrodes (12-18) of a pair, wherein the electrodes (12-18) are designed such that, when the measuring device is placed on a wearer, the electric current between the electrodes (12-18) of a pair flows through the body of the wearer underneath the skin. The compression therapy is monitored here by monitoring, through determination of impedance, the decrease in liquid in tissues that are treated by the compression.

The invention relates to an appliance for monitoring the compression therapy of a compression means comprising a measuring device made of a textile material to be applied to a wearer having two end sections, which enclose a middle section between them.

In the prior art, compression dressings are applied to body parts to treat edema and, for example, chronic venous insufficiencies. In this case, compression wrappings and also compression bandages or compression stockings are known, for example.

Compression dressings offer the advantage over compression bandages that the therapeutic dose of the pressure can be corrected during the application and the dressing can be continuously adapted to the affected body part upon reduction of the swelling.

However, most compression measures, in particular compression dressings, have the disadvantage that no information is received about the pressure during the application or the success of the compression measure, in particular no information is received with respect to the filtration and drainage in the capillaries. The therapy is thus difficult to monitor and incorrect application, namely either with too much or too little pressure, is unfortunately typical.

Users and patients are not capable of judging how good the course of therapy is, so that no information is provided, for example, about worsening of the health status or the respective therapy success.

In principle, vascular diseases are very well classified. The effectiveness of compression in these diseases is proven. However, possibilities for monitoring during the therapy are lacking.

It is therefore the object of the invention to provide an appliance and a method which enable monitoring of the compression therapy, not only as known in the past, by measuring the pressure, but rather by monitoring the liquid decrease in tissues treated by compression, since this is the therapeutically desired measure.

For example, devices are known from the prior art which determine the compression. Thus, for example, WO 2019/008376 discloses a method in which a first conductive path is provided in a wrapping as well as a sensor which measures the impedance of the wrapping.

The invention achieves this object by way of an appliance having the features of claim 1 and a method having the features of claim 9.

It is provided according to the invention that two electrodes are arranged in each end section, wherein one electrode forms an electrode pair with an electrode of the other end section in each case, wherein an electrical current flow is applied via the one electrode pair and an electrical voltage between the electrodes of a pair is determined via the other electrode pair, wherein the electrodes are designed so that the electrical current, with the measuring device applied to a wearer, flows between the electrodes of a pair through the body of the wearer below the skin or not through the skin.

By providing two electrode pairs, wherein one electrode of a pair is arranged in each case in a respective end section, it is possible to detect voltage and current over the extension of the measuring device, which can both be represented as a sine function.

The (bio-)impedance may be used here to determine the liquid content in a body of a wearer, which can be ascertained via the phase shift between the sine curve of the voltage and the sine curve of the current, which correlates with the liquid content in the monitored body section.

The electrodes are to be designed here so that the current flows through the body of the wearer below the skin and not solely in the region of the skin. The current flow is to take place in the intracellular and extracellular tissue. The impedance is the value by means of which a capillary pressure may be determined, which is considered in phlebology and lymphology to be an important parameter for biofiltration and drainage. The capillary pressure describes the interface pressure between the static fluids of the vascular system and the vascular wall here. A pressure increase on the wall from the outside would induce a fluid displacement from one compartment into the next, wherein this fluid displacement can be represented via the electrical impedance. The electrical impedance is represented here by the function

z=Rz+j lm

For lm=f(Re), the amplitude z of the sinusoidal voltage for a circuit is dependent on Θand describes the phase relationship between the voltage U(V) and the current I(A).

The human body or a limb can be hypothetically viewed as a capacitive resistor. The bodily fluids (electrolytes) permit a current flow. This means that a body part which is connected to an electrical circuit wherein, when the body part loses liquid due to compression (P in mmHg), this can be established by a variation of the electrical impedance (z), since the current flow (I) in this circuit is mathematically proportional to the voltage (V).

The mathematical formula here is

Z=R−j*X ,

wherein this is the capacitive bioimpedance here. P (mmHg) is the interface pressure below the compression means.

The mathematical model with respect to the interface pressure can be seen in FIG. 2. It can be inferred therefrom that the impedance decreases with increasing interface pressure.

A direct current source can preferably be used as the current source, for example, a battery or the like, since the movement of a wearer is not restricted too greatly in this way and the space requirement and the mobility of the usability is increased. An oscillator is arranged here between direct current source and electrode pair, to thus keep the amplitude of the current and the voltage stable and enable an impedance measurement.

An evaluation device is preferably provided, which determines the impedance of the body of a wearer via the phase shift, wherein the change of the liquid content in the observed body area induces the phase shift.

Furthermore, it is preferred if the textile material of the measuring device is a woven material and the electrodes are in particular woven electrodes. Preferably, these can be textile polymer electrodes. A coating can be applied in the electrode region which improves the conductivity, for example, made of electrically conductive silicone or a graphite-based paste. In woven electrodes, as are preferably to be used, the contact surface to the skin of the wearer is important, to prevent so-called “skin effects” from occurring, thus the current and the voltage not reaching the intracellular or extracellular region below the skin, but rather being discharged via the skin. The following parameters are relevant for the selection and design of the woven electrodes.

The technical variation Tv determines the effectiveness of the electrode with respect to the ion transport.

$$Tv = \frac{nWeft}{Se} \qquad Equation 1$$

nWeft is the number of the weft threads
S is the electrical surface contact of the electrode

The technical variation Tv is linked here to the parameters reed width and crimp percentage (crimp factor), so that the least possible skin effects occur.

The reed can be described indirectly by the mathematical equation of the width of the reed, since it enables the variation of the technical parameter. The reed width is described by the following equation:

Rw=Fwidth−(Fwidth*Weft crimp factor) Equation 2

Rw is the reed width. The reed is described with respect to its width according to equation 2 by Fwidth.

Fwidth is the width of the woven planar formation. Since the electrode is a part of the planar formation, the width of the planar formation influences the technical variation of the electrode, namely the electrical surface contact. In this case, the electrodes are woven in the weft direction (thus the width) of the planar formation to provide their function for the impedance measurement. There is therefore a relationship between the width of the planar formation and the desired size of the electrode. In addition to the textile width, the way in which the threads are incorporated in the width (crimp) has an influence.

During the creation of the planar formation, the warp threads, depending on their tension and the selected binding, cause a weft thread crimp. The weft thread crimp is described by the weft crimp factor. Weft crimp factor is the factor with respect to the length change of the weft in the planar formation.

The surface contact is preferably correlated here in a strongly negative manner with the warp crimp factor (Warp Cf). The warp crimp factor describes the warp thread crimp here.

$$warp crimp factor \\ Warp Cf = \frac{Warp length - Fabric length}{Fabric length} \qquad Equation 3$$

In principle, the surface contact can be described by the warp crimp factor in the following equation:

$$surface contact Knowing that \\ Tv = \frac{nWeft}{Se} ↔ Se = \frac{nWeft}{Tv} , \qquad Equation 4$$

due to the equivalence, equation 4 results:

$$nWef * WarpCf = Tv * nWeft \\ ↔ Tv = Warp Cf \\ \to Se = \frac{nWeft}{Warp Cf}$$

It can particularly preferably be provided here that the appliance is a wrapping, in particular a compression wrapping. Alternatively, the appliance can also be designed as a (compression) stocking or as a sleeve with or without compression effect. If the appliance itself is capable of applying the compression, a further additional compression appliance in the form of a wrapping, for example, can be saved.

The electrodes of one end section are preferably arranged one over the other in the weft direction and have a preferred distance d≥5 cm. The distance to the edge of the end section is in particular ≥1 cm.

In a wrapping/bandage, the weft direction corresponds here to the transverse direction and the warp direction to the longitudinal direction.

It has proven to be particularly expedient if the electrodes can be contacted via electrically conductive threads, which are in particular guided in the textile material and are in particular connected via this to the current source and/or the evaluation device. In this way, the contacting takes place without more cable than required having to be provided for the contacting.

According to the invention, one or both end sections can preferably be made inelastic and the middle section can be made elastic in order to be able to better design the electrodes in the end sections and determine the contact surface with the wearer in a defined manner. The applicability can be improved and a compression can be achieved via the elastic formation of the middle section. The stretchability and elasticity is adjustable in a conventional manner and can be designed as a long-stretch wrapping or short-stretch wrapping. The above statement applies similarly in the design as a sleeve or stocking, so that here the regions of the electrodes are inelastic and the part arranged in between in the longitudinal direction of the limbs is elastically stretchable. The stretchability is preferably in the range of 40% to 100% of the unstretched length. The stretchability is ascertained here according to the following method based on DIN 61632. A tensile testing device can be used for ascertaining the stretch. A test specimen of the planar formation (clamping length 200 mm) is loaded by a tensile speed of 200 mm/minute to a maximum force of 3 N/cm width.

The test specimen is laid before beginning measurement for at least 15 minutes without tension on a smooth, flat underlying surface (for example a table), it is subsequently clamped in the tensile testing device and stretched. The tensile testing device ascertains the stretch and uses the unstretched length (L0) and (L1). L0 is the initial length of the test specimen at the start and L1 is the length of the maximum force.

$$ε \% = ( \frac{L 1}{L 0} - 1 ) * 100 \qquad Equation 5$$

The electrically conductive threads have to be introduced so that they do not obstruct the stretchability.

To improve the conductivity and the introduction of the current into the skin, an electrically conductive coating can be applied in the region of the electrodes. This consists, for example, of an electrically conductive silicone or a graphite paste. The irregularities in the surface can also be compensated for in this way, which result due to the up-and-down movement of the thread in woven electrodes and due to which an influence on the contact surface results. The design of the electrodes, for example, with respect to the density of the weave, can vary depending on whether a coating is provided.

The invention also relates to a method for determining the compression of a compression means by means of a measuring device made of a textile material, wherein the measuring device is applied to a body, in particular a limb of a wearer, so that two end sections of the measuring device accommodate a region of a body to be measured between them, wherein each end section has two electrodes which each interact in pairs with the electrodes of the other end section and wherein an electrical current flow between the electrodes of one pair is determined via the one electrode pair and an electrical voltage between the electrodes of one pair is determined via the other electrode pair, wherein the electrical current flows, when the measuring device is applied to a wearer, between the electrodes of a pair through the body of the wearer below the skin.

In the method, the bioimpedance of the body is preferably determined via the phase shift.

It is particularly preferred here if the measuring device is wound as a wrapping on the body and is also preferably furthermore used as a compression means.

The determination of the qualitative change of the liquid content of a body both with respect to the intracellular and also extracellular liquid was carried out in experiments to confirm the function of the specified correlation between effect of the compression and thus the transport (away) of liquid from the body and impedance. An appliance in the form of a bandage was wound onto a leg of a wearer used as a test subject, wherein two bandage layers overlapped in each case.

The stretching of the bandage was approximately 110% of the original length. Measurements were made in recumbent, seated, and standing position. The test subjects were healthy. The electrodes were not part of that appliance but rather were applied as adhesive electrodes to the skin of the test subjects.

Before the experiment, the test subjects rested for approximately 3 minutes in the recumbent position. After each body movement, the body was given a minute of relaxation before the measurement.

The impedance was measured before the compression. Impedance and pressure were measured with applied bandage in the three positions. The test subject then wore the bandage for several hours during their activities typical in this time, for example, professional activities.

A final measurement took place after the compression was reduced and was compared to the values without compression.

Test results:

Impedance of the limbs before the compression:

| | R (z) | X (z) | z (Ω) | P (mmHg) | n |
|---|---|---|---|---|---|
| Test | 216 | 20 | 217 | | 9 |
| subject | | | | | |
| 1 | | | | | |
| Test | 253 | 32 | 255 | | 9 |
| subject | | | | | |
| 2 | | | | | |

Static results in consideration of the mean value of all positions (recumbent, seated, standing):

a) Impedance and pressure at the point in time t=0

| | R (z) | X (z) | z (Ω) | P (mmHg) | n |
|---|---|---|---|---|---|
| Test | 233 | 22 | 234 | 29 | 18 |
| subject | | | | | |
| 1 | | | | | |
| | 241 | 21 | 241 | 52 | 9 |
| Mean | 237 | 21 | 238 | 41 | |
| value | | | | | |

b) Impedance and pressure at the point in time t=1.5 hours

| | R (z) | X (z) | z (Ω) | P (mmHg) | n |
|---|---|---|---|---|---|
| Test | 232 | 20 | 233 | 38 | 18 |
| subject | | | | | |
| 1 | | | | | |
| | 244 | 23 | 245 | 45 | 9 |
| Mean | 238 | 22 | 239 | 41 | |
| value | | | | | |

Static results in consideration of the mean value of all positions (recumbent, seated, standing):

a) Impedance and pressure at the point in time t=0

| | R (z) | X (z) | z (Ω) | P (mmHg) | n |
|---|---|---|---|---|---|
| Test | 256 | 32 | 258 | 18 | 9 |
| subject | | | | | |
| 2 | | | | | |
| | 262 | 30 | 264 | 40 | 9 |
| Mean | 259 | 31 | 261 | 29 | |
| value | | | | | |

b) Impedance and pressure at the point in time t=1.5 hours

| | R (z) | X (z) | z (Ω) | P (mmHg) | n |
|---|---|---|---|---|---|
| Test | 238 | 23 | 239 | 17 | 9 |
| subject | | | | | |
| 2 | | | | | |
| | 256 | 27 | 258 | 35 | 9 |
| Mean | 247 | 25 | 248 | 26 | |
| value | | | | | |

Impedance of the limbs with significant reduction of the compression

| | R (z) | X (z) | z (Ω) | P (mmHg) | n |
|---|---|---|---|---|---|
| Test | 234 | 23 | 235 | | 9 |
| subject | | | | | |
| 1 | | | | | |
| Test | 240 | 24 | 241 | | 9 |
| subject | | | | | |
| 2 | | | | | |

Summary of the results:

| | Test subject 1 | | Test subject 2 | |
|---|---|---|---|---|
| | z (Ω) | P (mmHg) | z (Ω) | P (mmHg) |
| Beforehand | 217 | 0 | 255 | 0 |
| Compression | 238 | 41 | 261 | 29 |
| Compression | 239 | 41 | 248 | 26 |
| End | 235 | 18 | 241 | 18 |
| compression | | | | |

The result is shown hereafter in FIG. 6, which shows that the impedance without compression is initially low and then increases with the compression and then drops again when the compression diminishes. This correlates with the liquid transport which is stimulated by the compression. If the compression diminishes, the liquid transport in the cells and the extracellular transport also ends.

The invention is explained in more detail hereinafter on the basis of a drawing. In the figures:

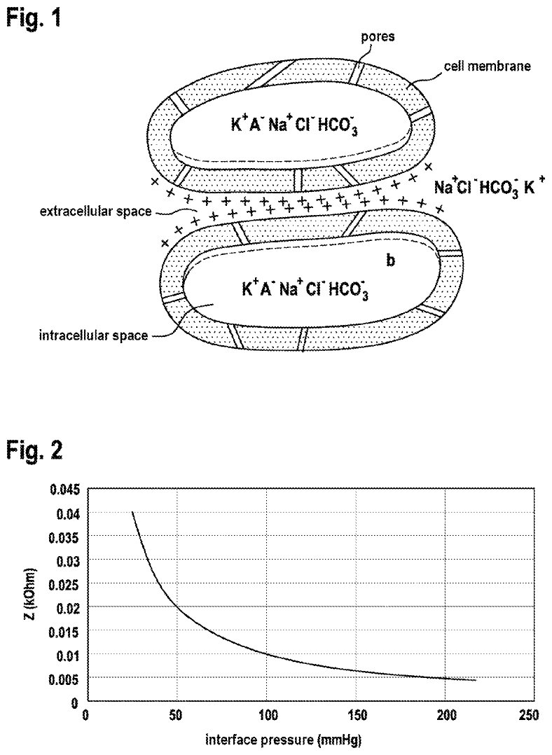

FIG. 1 shows a so-called body hydration model;

FIG. 2 shows a mathematical model;

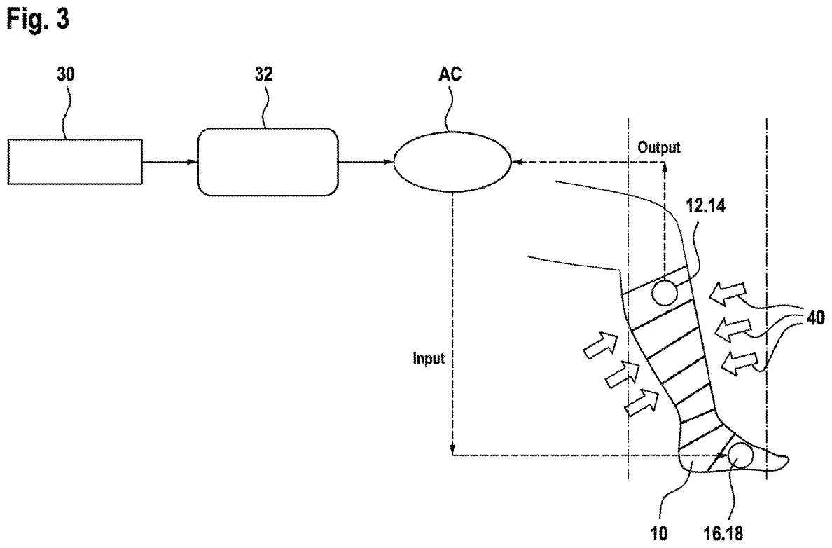

FIG. 3 shows an appliance according to the invention in the form of a compression bandage;

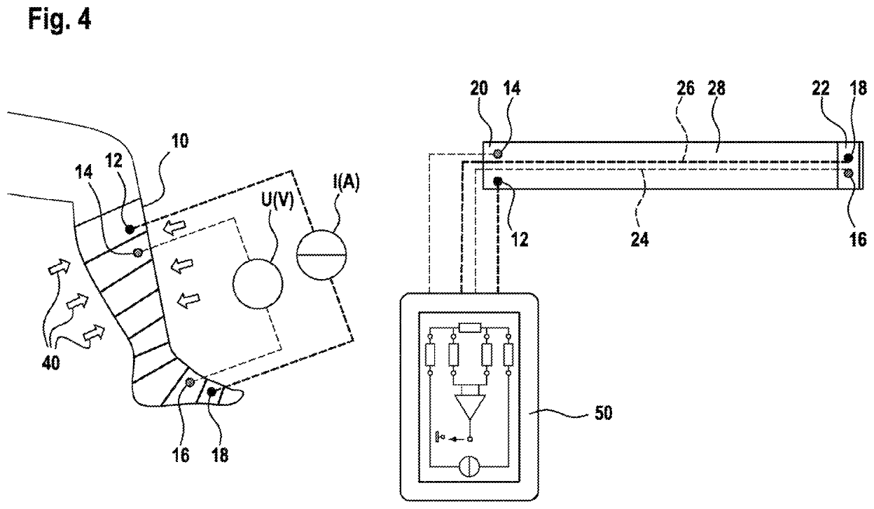

FIG. 4 shows a further representation of the invention;

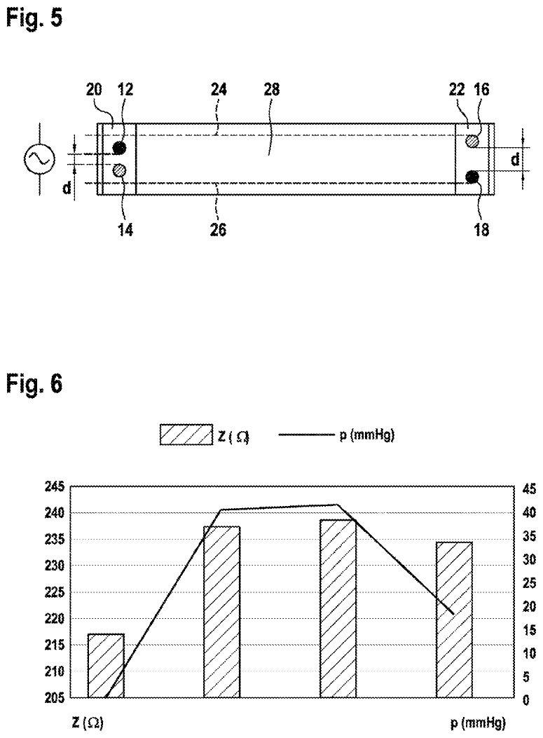

FIG. 5 shows a bandage according to the invention; and

FIG. 6 shows a representation of the experimental result.

FIG. 1 shows a body hydration model, which represents how the liquid is distributed in the body, namely both in the intracellular space and also in the extracellular space, thus between the individual cells. The intracellular liquid can enter through the pores in the cell membranes into the extracellular space and can be transported further from there.

FIG. 2 shows the dependence of the bioimpedance on the interface pressure and thus the compression as explained above. A patient having edema therefore has a lower impedance value Z before the therapy than during the compression therapy. If, for example, a venous insufficiency is treated using a compression pressure of approximately 50 mmHg in a typical manner, it is to be expected that the patient will have a decreasing impedance value when the compression pressure is increased, for example, to 80 mmHg. Monitoring the bioimpedance therefore supplies information about the transport of the liquids away in the intracellular and extracellular space.

FIG. 3 shows a first design of the appliance 10 comprising a measuring device for a compression means, which is designed here as a wrapping and at the same time forms the compression means. In two end sections, which are shown in more detail in FIG. 5, two electrodes are provided here in each case, wherein the current is provided as direct current via a battery 30 in order to restrict the mobility of the wearer as little as possible. The direct current is converted in an oscillator 32 so that the amplitude remains constant. The current is then conducted via the skin of the wearer into the body so that the current flow through the limbs and not through the skin can be determined. The compression effect is indicated at the same time by arrows 40. In addition to the input, the output of both the current and also the electrical voltage is also measured. The evaluation device bears the reference sign 50. This is also shown in particular in FIG. 4, where both the current measurement I and also the voltage measurement U are symbolized. The bioimpedance through the limb can be determined via this.

FIG. 4 shows a bandage according to the invention or also a wrapping, which is used synonymously, applied to a leg of a wearer in the context of the application, in which woven electrodes 12, 14, 16, 18 are provided at a first end section 20 and a second end section 22, wherein each end section 20 has two electrodes 12, 14, 16, 18 and these each form a pair with one electrode 12-18 of the other end section 22. The activation or the current introduction into the electrodes 12-18 takes place by means of electrically conductive threads 24, 26, which are woven into the bandage/wrapping.

The woven electrodes 12-18 also consist here of electrically conductive threads. The end sections 20, 22 are one or both formed from an inelastic textile, wherein the middle section 28 arranged in between is made elastically stretchable and thus also permits the use not only as a measuring device, but also as a compression means. The threads 24, 26 are woven in so that the elasticity is not impaired.

To achieve a better transition of the current into the body, it can be provided that a coating is provided to compensate for irregularities in the region of the electrodes. This can consist of or comprise an electrically conductive silicone or a graphite paste. In this way, the contact surface with the body of the wearer is enlarged.

The distance d between the electrodes of an end section 20, 22 is at least 5 cm here.

The warp thread direction preferably corresponds to the wrapping longitudinal direction and the weft thread direction corresponds to the transverse extension of the wrapping.

The inelastic end sections 20, 22 can be made inelastic here according to one design via a coating, special thread selection, or by the technical variation Tv.

FIG. 6 shows once again, as explained above, the result of the test.

1. An appliance for monitoring the compression therapy of a compression means comprising a measuring device made of a textile material to be applied to a wearer having two end sections, which enclose a middle section between them, and wherein, two electrodes are arranged in each end section, which each form an electrode pair with one electrode of the other end section, wherein an electrical current flow is determined via the one electrode pair and an electrical voltage is determined via the other electrode pair between the electrodes of one pair, wherein the electrodes are designed so that the electric current, when the measuring device is applied to a wearer, flows between the electrodes of one pair through the body of the wearer below the skin.

2. The appliance as claimed in claim 1, wherein a direct current source is used as the current source, and wherein an oscillator is arranged between the direct current source and the electrode pair.

3. The appliance as claimed in claim 1 further comprising an evaluation device, which determines the impedance of the body of a wearer via a phase shift.

4. The appliance as claimed in claim 1, wherein the textile material of the measuring device is a woven material and the electrodes are woven electrodes.

5. The appliance as claimed in claim 1 wherein the measuring device is a wrapping.

6. The appliance of claim 1, wherein the electrodes can be contacted via electrically conductive threads.

7. The appliance of claim 1, wherein the end sections are inelastic and the middle section is elastic.

8. The appliance of claim 1, wherein an electrically conductive coating is applied in the region of the electrodes.

9. A method for determining the compression of a compression means by means of a measuring device made of a textile material, wherein the measuring device is applied to a body, so that two end sections of the measuring device accommodate a region of a body to be measured between them, wherein each end section has two electrodes, which each interact with the electrodes of the other end section in pairs, and wherein an electrical current flow between the electrodes of a pair is determined via the one electrode pair and an electrical voltage between the electrodes of a pair is determined via the other electrode pair, wherein the electrical current, when the measuring device is applied to a wearer, flows between the electrodes of a pair through the body of the wearer below the skin.

10. The method of claim 9, wherein the bioimpedance of the body is determined via a phase shift.

11. The method of claim 9, wherein the measuring device is wound onto the body as a bandage.

12. The appliance of claim 5, wherein the wrapping is a compression wrapping.

13. The appliance of claim 6, wherein the electrically conductive threads are guided in the textile material.

14. The appliance of claim 13, wherein the electrically conductive threads are guided in the textile material and are connected via this guiding to a current source and/or an evaluation device.

15. The method of claim 9, wherein the body to which the measuring device is applied is a limb of a wearer.
